(19) United States
(12) Patent Application Publication
(10) Pub. No.: US 2007/0072948 A1
(43) Pub. Date: Mar. 29, 2007

(54) Composition comprising long-chain alcohols for suppressing concentrations of C-reactive protein (CRP) in human blood

(75) Inventors: Bernhard Kammermeier, Munchen (DE); Franz-Leo Heinrichs, Gablingen (DE); Ernst Krendlinger, Friedberg (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant Produkte (Deutschland) GmbH

(21) Appl. No.: 11/522,146

(22) Filed: Sep. 15, 2006

(30) Foreign Application Priority Data
Sep. 24, 2005 (DE) 10200504829.7

(51) Int. Cl. A61K 31/045

(52) U.S. Cl. 514/724

(57) ABSTRACT

Long-chain alcohols based on montan wax alcohols, beeswax alcohols and Guerbet alcohols can advantageously be used in a composition for efficiently lowering elevated CRP levels. It is surprisingly also possible thereby to influence beneficially pathophysiological processes, in particular CHD, stroke or complications of rheumatoid diseases which are associated with elevated CRP levels.

The invention relates to the use of long-chain alcohols in a composition for efficiently reducing pathologically elevated CRP levels.

C-reactive protein (CRP) is a carbohydrate-free factor which is produced in the liver and occurs in association with inflammatory processes, both mild and severe, local as well as generalized. The name derives from its ability to react in the presence of Ca ions with a pneumococcal polysaccharide. CRP is one of the acute phase proteins and is able to activate the complement system. Its production in the liver is stimulated by interleukin 6 (see “http://de.wikipedia.org/wiki/C-Reaktives-Protein”).

It is known that elevated CRP levels represent an independent risk factor for life-threatening cardiovascular diseases, or for stroke (New England Journal of Medicine, 347, 2002, 1557). Recent research also attributes CRP with the role of a risk factor for arteriosclerosis (see “http://www.ilexikon.com/C-Reaktives-Protein.html”).

It is further known that statins lower CRP even in the absence of a hyperlipidemia (Journal of American Medical Association, 2002, 286:64-70) and thus CRP is possibly a stronger predictive factor in relation to the risk of CHD than are elevated LDL levels (see http://www.aerztezeitung.de/docs/2002/11/18/208a 11.asp?cat=/medizin/cholesterin).

It is additionally known that elevated CRP levels are also involved in rheumatoid diseases. CRP makes it possible in rheumatology to distinguish between inflammatory rheumatoid diseases and non-inflammatory rheumatoid diseases (see http://www.rheuma-online.de/a-z/c/c-reaktives-protein.html). CRP is in this connection for example rapidly influenced by cortisone. Influencing the inflammatory markers CRP and SAA (serum amyloid A protein) through administration of folates is also known (see WO 03/072096).

It was therefore an object of the present invention to find a composition with which the CRP level in the blood can be lowered and with which therefore all the abovementioned pathologies associated with a high CRP level can be beneficially influenced, mainly on the beneficial influencing of cardiovascular complications via a reduction in the CRP concentration.

This object is achieved by the use of long-chain alcohols based on montan wax alcohols, beeswax alcohols and Guerbet alcohols in a composition for efficiently lowering elevated CRP levels.

Long-chain alcohols like those occurring in the abovementioned montan wax alcohols, beeswax alcohols and Guerbet alcohols are known to have cholesterol-lowering effects in analogy to statins (see E. Ernst, MMW No. 23/2002, page 20, or WO 00/78 697 A2). Animal experimental investigations have revealed in this connection that this mixture of alcohol substances has, besides the effects mentioned, also further antiatherogenic and other beneficial health effects, with no adverse effects having been found. Comparative studies show an effect equivalent to conventional statins (simvastatin and pravastatin were compared) with a lack of a side effect profile in a three-year comparison.

The pathophysiological correlation of elevated LDL and elevated CRP levels makes it possible to conclude that there is a physiological interaction of the LDL and CRP levels.

It has been found, entirely surprisingly, that the use of long-chain alcohols based on montan wax alcohols, their beeswax analogs and Guerbet alcohols in a composition is outstandingly suitable for efficiently lowering pathologically elevated CRP levels and thus that it is possible to influence beneficially pathophysiological processes associated with elevated CRP levels.

No specific alcohol fractions are removed from the aforementioned long-chain alcohols; on the contrary, the respective entire alcohol contents, prepurified where appropriate, are used.

The long-chain unbranched alcohols to be derived from the abovementioned wax alcohols, and the Guerbet alcohols advantageously show a far simpler, more cost-effective and in some cases also stronger effect in relation to suppressing the CRP level than the known statins.

The known wax alcohols additionally prove to be non-toxic as long-chain, unbranched or branched alcohols in industrial toxicology studies (see: “Montan Wax” article in: Ullmann's Encyclopedia of Industrial Chemistry, Vol A 28, 5th edition, Weinheim, Verlag Chemie, pp. 122-126). The great advantage of said alcohols derives from their simple industrial synthesis and their qualitative reproducibility and availability.

The alcohols can be cleaved from the wax by hydrolysis reactions and subsequently be extracted for purification. The montan wax can also initially be cleaved by oxidation, e.g. with chromic acid (so-called Gersthofen process), and the resulting montan wax acids can then be converted into alcohols by reduction. Guerbet alcohols can likewise be obtained from readily obtainable raw materials by a very simple chemical reaction.

The long-chain alcohols are employed according to the invention in amounts in the range from 0.01 to 3% by weight based on the patient's body weight, preferably from 0.1 to 2% by weight. Administration preferably takes place orally, but can also with an appropriate formulation take place rectally, subcutaneously or intravenously.

1. A composition for efficiently lowering elevated CRP levels comprising one or more long-chain alcohols selected from the group consisting of montan wax alcohols, beeswax alcohols. Guerbet alcohols and mixtures thereof.

2. A composition for treating coronary heart disease, stroke or complications of rheumatoid diseases comprising one or more long-chain alcohols selected from the group consisting of montan wax alcohols, beeswax alcohols, Guerbet alcohols and mixtures thereof.

3. A composition for avoiding rhabdomyelosis complications comprising one or more long-chain alcohols selected from the group consisting of montan wax alcohols, beeswax alcohols, Guerbet alcohols and mixtures thereof.

4. The composition as claimed in claim 1, wherein the one or more long chain alcohols are present in a therapeutically acceptable amount determined by a well-established analytical method.

5. The composition as claimed in claim 1, wherein, when administered, produces no additional caloric load.

6. The composition as claimed in claim 1, wherein the one or more long-chain alcohols are employed from 0.01 to 3% by weight based on the patient's weight.

7. The composition as claimed in claim 1, wherein the composition is a composition for oral administration.
